(19) United States
(12) Patent Application Publication
(10) Pub. No.: US 2014/0276667 A1
(43) Pub. Date: Sep. 18, 2014

(54) TELESCOPING MEDICAL INSTRUMET

(71) Applicant: TransEnterix Surgical, Inc., Morrisville, NC (US)

(72) Inventors: Carson Shellenberger, Raleigh, NC (US); Nicholas J Bender, Raleigh, NC (US)

(73) Assignee: TransEnterix Surgical, Inc., Morrisville, NC (US)

(21) Appl. No.: 14/216,713

(22) Filed: Mar. 17, 2014

Related U.S. Application Data
(60) Provisional application No. 61/801,499, filed on Mar. 15, 2013.

(51) Int. Cl. A61B 17/00

(52) U.S. Cl. CPC A61B 17/00234
USPC 606/1

(57) ABSTRACT

An instrument shaft includes an outer elongate shaft having a rigid proximal section, a rigid distal section, and a fixed bend between the rigid proximal and distal sections. An inner shaft includes rigid proximal and distal sections and a flexible intermediate section between the rigid proximal and distal sections. The inner shaft is slidably disposed within the lumen of the outer shaft such that the flexible intermediate section extends through the bend.

This application claims the benefit of U.S. Provisional Application No. 61/801,499, filed Mar. 15, 2013, which is incorporated herein by reference.

### FIELD OF THE INVENTION

The present invention relates generally to the field of surgical instruments. More particularly, the present invention relates to instruments having distal portions that are longitudinally extendable within a body cavity.

### BACKGROUND

A surgical grasper is a conventional instrument used in open surgical procedures, as well as in less invasive procedures such as laparoscopy, single port surgery, and natural orifice procedures. A conventional grasper includes a straight rigid shaft with a pair of jaws on its distal end and a handle on the proximal end. During use, when it is desired to extend the jaws further into the body cavity, the user moves the handle towards the patient, moving the entire instrument in a distal direction.

In minimally invasive procedures, multiple instruments may be passed into a body cavity via a common incision, typically through an access port disposed within the incision. It is desirable to configure the instruments in a manner that avoids conflict between the various instrument handles extending proximally from the access port. This application describes an instrument shaft assembly configured to minimize conflict between instrument handles.

### BRIEF DESCRIPTION OF THE DRAWINGS

FIG. 1 is a side elevation view of an articulating grasper, in which the outer shaft is shown transparent to allow the inner shaft to be seen;

FIGS. 2A and 2B are side elevation views of the grasper of FIG. 1, in which FIG. 2A shows the distal end of the shaft in a proximal position and in which FIG. 2B shows the distal end of the shaft in a distal position;

FIG. 3 is a longitudinal cross-section view of a portion of the shaft of FIG. 1;

FIGS. 4A and 4B show the instrument extending through an access port (only the proximal portion of which is shown) positionable in an incision into a body cavity. A rigid scope is also shown extending through the access port. FIG. 4A shows the end effector positioned in an extended position relative to FIG. 4B.

### DETAILED DESCRIPTION

This application describes an instrument shaft suitable for use on surgical instruments, particularly those intended for use in combination with scopes and/or other instruments that are advanced into the body via a common incision or access port. In a first embodiment, the shaft is described as the shaft of a rigid grasper. In a second embodiment, the shaft , the shaft forms part of a steerable or articulatable instrument channel device that allows flexible instruments to be passed through its lumen. However, it should be appreciated that the shaft can be incorporated into other forms of surgical devices using different end effectors and handle arrangements.

### First Embodiment

Referring to FIG. 1, instrument 10 includes an elongate shaft generally indicated by reference number 20. The shaft 20 has a handle 30 at its proximal end and an end effector 40 on its distal end. In this embodiment, the end effector comprises grasper jaws.

Shaft 20 includes a rigid outer tubular shaft 12. Outer shaft 12 has straight proximal and distal sections 13a, 13b joined by a bend 14, which may comprise an angled or curved section 14. The shape of the shaft 20 is fixed, such that its shape cannot be changed during ordinary use of the instrument. In the illustrated embodiment, the straight distal section 13b of the shaft 12 defines a longitudinal axis of the instrument 10, whereas the straight proximal section 13a has an axis transecting the longitudinal axis.

An inner shaft, generally indicated by numeral 16, is disposed in the lumen of the rigid outer shaft 12. It comprises a rigid proximal section 18a, a flexible intermediate section 18b, and a rigid distal section 18c. In one embodiment, the rigid sections 18a, 18c are formed of rigid tubing having a fixed straight shape that cannot be changed during ordinary use of the instrument. The flexible section 18b is formed of coiled pipe or other material having sufficient flexibility to traverse the bend 14 within the outer shaft 12.

Inner shaft 16 is positioned such that the flexible section 18b extends through the bend 14 of the outer shaft 12. A portion of rigid distal section 18c is disposed in distal section 13b of the outer shaft, and thus in the illustrated embodiment has as its axis the longitudinal axis of the instrument. A portion of rigid proximal section 18a is disposed in proximal section 13a of outer shaft 12, and thus extends angularly from the longitudinal axis of the instrument.

The end effector 40 is mounted to the distal section 18c of the inner shaft. The handle 30 is mounted to the proximal section 18a of the inner shaft and thus in the shown embodiment is offset from the longitudinal axis of the instrument due to the angled proximal section 13a of the outer shaft.

The inner shaft 16 is slidable within the lumen of the outer shaft 12 to advance/retract the end effector 40 relative to the outer shaft 12. In particular, the inner shaft is moveable from a first position such as that shown in FIG. 2A, in which the end effector extends from the distal end of the outer shaft by a first distance, to a second position (FIG. 2B) in which the end effector extends from the distal end of the outer shaft by a second distance which is greater than the first distance. Note that movement of the inner shaft from the first position to the second position is accomplished by moving the handle 30 distally, and thus closer to the proximal end of the outer shaft 12.

The inner shaft 16 may include a lubricious outer surface (on its full length, or on just the portion that slides within the outer shaft), formed by coating or applying a sleeve to the shaft, to facilitate movement of the inner shaft 16 through the lumen of the outer shaft 12. Alternatively, or additionally, the walls of the inner shaft's lumen may be made lubricious in a similar way. Materials suitable for covering or coating the outer shaft lumen and/or the inner shaft surface include but are not limited PTFE.

Referring to FIG. 3, a cable extends through the shaft 20 from the handle 30 to the end effector 40, such that manipulation of the handle actuates opening/closing of the jaws in a manner known to those skilled in the art. In one embodiment, the cable includes a flexible intermediate section 52b extending through the flexible intermediate section 18b of the shaft. Proximal and distal sections 52a, 52c of the shaft may be stiff or rigid, or they may also be flexible.

FIGS. 4A and 4B illustrate use of the device in combination with an access device 60 disposed in an incision (not shown) formed in a body cavity such as the abdominal cavity. The access device may be any of a variety of access devices or ports suitable for laparoscopic or single incision surgery. The access device may be a type having an elongate tube that extends into the body, and one or more sealed ports at its proximal end. The seals are positioned to allow insufflation pressure to be maintained within the abdominal cavity during use of instruments through the ports, and when the ports do not have instruments through them. The illustrated access device 60 includes multiple such ports 62 for independently receiving different instruments. In the drawing, only a proximal portion of the access device 60, with the sealed ports 62 is shown. These figures further show a second instrument, in this case a rigid scope 64, extending through one of the ports 62 of the access device.

In use, the distal end of the instrument 10 is inserted through a port 62 in the access device to position the end effector 40 within the body cavity, while the handle 30 remains outside the body. The distal end of the second instrument is likewise inserted through a port in the access device. A rotational orientation of the shaft 20 may be chosen to position the handle 30 such that it extends away from the proximal end of the second instrument to avoid conflict between the two.

The longitudinal position of the rigid outer shaft 12 relative to the access device is preferably maintained throughout out the procedure. This may be by friction between the outer shaft 12 and the access device, or by a latch used to engage the outer shaft 12 to the access device or to a support arm that supports the outer shaft 12 in a fixed position relative to the patient. To change the longitudinal position of the end effector 40 within the patient, the handle 30 is pushed or withdrawn to advance or withdraw the inner shaft 16 relative to the outer shaft 12. To longitudinally advance the end effector within the body cavity, the handle may be advanced to the position shown in FIG. 4A, or still further until the handle contacts the outer shaft 12 as in FIG. 2B. To longitudinally withdraw the position of the end effector 40, the handle may be withdrawn as shown in FIG. 4B or 2A. As seen in the drawings, the amount of the proximal and distal rigid sections 18a, 18c of the inner shaft 16 extending from the proximal and distal ends, respectively, of the outer shaft 12 varies with the position of the inner shaft 16.

The disclosed instrument shaft is beneficial in that it allows the clear space just proximal to the angled proximal section 13a to be maintained regardless of the longitudinal position of the instrument, because the position of the bend 14 does not change when longitudinal adjustments are made to the instrument's position. More particularly, if moving the end effector from the FIG. 4A position to the FIG. 4B position required proximal movement of the bend 14, the bend would encroach or even collide with the scope at the proximal end of the scope. Instead, the region surrounding the scope (illustrated using a circle in FIG. 4B), remains clear even with the instrument is moved proximally.

Moreover, when the instrument is used extending through sealed ports in an access device, the end effector may be advanced/retracted without sliding the instrument shaft back and forth through the seals in the ports 62 of the access device, thus preserving the seals and helping abdominal insufflation pressure to be maintained.

The instrument may be provided with instructions for use instructing the user to extend the shaft using methods described herein or equivalent thereto.

### Second Embodiment

In a second embodiment, the instrument shaft is incorporated into an access device or a steerable channel device—allowing flexible instruments to be passed through the lumen of the inner shaft such that the end effectors of those flexible instruments are positioned in a body cavity. Such designs include means for steering or articulating the distal end of the inner tube in order to steer or articulate the end effector. For example, referring to FIG. 1, in the second embodiment, the end effector 40 is replaced by an additional tubular section on the distal end of the distal section 18c. The additional section may be a bendable or flexible section comprised of vertebrae, slotted tubing, etc, which is steered using actuation elements (e.g. wires/cables etc.) engaged by an actuator moveable by a user to steer the bendable/flexible section. Steerable sections of this type, as well as mechanisms for steering them, are shown on steerable instrument channels in U.S. Publication No. 2011/0184231, which is incorporated herein by reference. The steerable sections might also be steered using motors to engage the actuation elements or pull cables extending through the steerable instrument channels in the manner described in commonly owned U.S. application Ser. No. 13/759,036, filed Feb. 4, 2013, which is incorporated herein by reference. In another embodiment, the additional section may be another rigid section pivotally connected to the distal section 18c and articulated by manually- or motor-driven actuation elements that extend through the inner shaft.

Using a steerable channel device having a telescoping shaft of the type disclosed herein, the steerable distal section may be advanced distally and proximally through the access device through which it is positioned, without disrupting sealing between the seal in the access devices port (see port 62 of FIGS. 4A and 4B). It should be noted that while in FIGS. 4A and 4B the bend 14 is positioned at a proximal location, the bend may be positioned towards the distal end of the shaft as well.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above, including for purposes of priority, are incorporated by reference.

We claim:

1. An instrument shaft comprising:
an outer elongate shaft having a rigid proximal section, a rigid distal section, and a fixed bend between the rigid proximal and distal sections, the outer shaft further including a lumen;
an inner elongate shaft having a rigid proximal section, a rigid distal section, and a flexible intermediate section between the rigid proximal and distal sections; wherein
the inner elongate shaft is disposed within the lumen of the outer elongate shaft such that a portion of its rigid distal section is disposed within the rigid distal section of the outer elongate shaft, such that a portion of its rigid proximal section is disposed within the rigid proximal section of the outer elongate shaft, and such that the flexible intermediate section extends through the bend;
the inner elongate shaft slidable within the lumen of the outer shaft between a first position in which a distal end of the outer shaft is separated from the distal end of the inner shaft by a first distance, and a second position in which a distal end of the outer shaft is separated from the distal end of the inner shaft by a second distance greater than the first distance.
